(19) United States
(12) Patent Application Publication
(10) Pub. No.: US 2026/0171102 A1
(43) Pub. Date: Jun. 18, 2026

(54) SYSTEMS, DEVICES, AND METHODS FOR GENERATING VOCAL DATA

(71) Applicant: Midjourney, Inc., South San Francisco, CA (US)

(72) Inventors: Brian A. KAPPUS, Campbell, CA (US); Ahmad ABBAS, South San Francisco, CA (US); Jackson OSWALT, South San Francisco, CA (US); David HOLZ, South San Francisco, CA (US)

(21) Appl. No.: 19/418,774

(22) Filed: Dec. 12, 2025

Related U.S. Application Data
(60) Provisional application No. 63/734,694, filed on Dec. 16, 2024.

(51) Int. Cl. G10L 21/02; H04R 19/04

(52) U.S. Cl. CPC G10L 21/02; H04R 19/04

(57) ABSTRACT

Devices, systems, and methods herein relate to the measurement of reflected acoustic waveforms to generate vocal data and avoid undesired noise, minimize calibration and set up, and improve continuous usability. These systems, devices, and methods may include transmitting an acoustic waveform through air to a skin of a subject, measuring a reflected acoustic waveform reflected by the skin of the subject, and processing the measured reflected acoustic waveform to generate vocal data of the subject.

### INCORPORATION BY REFERENCE

This application claims priority to U.S. Provisional Application Ser. No. 63/734,694 filed on Dec. 16, 2024, which is hereby incorporated by reference in its entirety.

### TECHNICAL FIELD

The devices, systems, and methods herein relate to a microphone.

### BACKGROUND

Traditional microphones are useful for capturing sound waves produced by a speaker (e.g., person), but are susceptible to picking up undesirable external noise (e.g., wind, vehicles, other speakers) while also struggling to record quieter voices in noisy environments. However, conventional solutions that attempt to capture voice while reducing external noise are frequently intrusive and uncomfortable. For example, to capture a speaker's voice, a contact throat microphone maintains contact with the skin of the neck to directly sense the physical vibration of the skin caused by movement of the vocal cords. However, the invasiveness and physical discomfort of contact throat microphones limits their practicality and broader adoption. As such, additional devices, systems, and methods for measuring vocal data in manner that avoids unwanted noise without the need to contact sensitive areas of the skin are desirable.

### SUMMARY

Devices, systems, and methods herein relate to the measurement of reflected acoustic waveforms to generate vocal data with improved noise immunity, minimal calibration and set up, and continuous and uninterrupted performance. For example, continuous waveforms may be transmitted to a speaking subject (e.g., speaker) in a non-contact manner such that their throat vibrations (corresponding to their voice) become coupled to the transmitted continuous waveform. The coupled waveforms may then be measured and processed to generate the vocal data. Advantageously, environmental noise (e.g., non-speaker noise) does not substantially interfere (e.g., couple) with the continuous waveform, thereby facilitating high quality vocal data. For example, a method of generating vocal data may include transmitting an acoustic waveform through air to a skin of a subject; measuring a reflected acoustic waveform reflected by the skin of the subject; and processing the measured reflected acoustic waveform to generate vocal data of the subject.

In some variations, the reflected acoustic waveform may include the acoustic waveform coupled with a voice signal corresponding to a vibration in the skin. In some variations, the reflected acoustic waveform may include a change in one or more of a phase and a frequency relative to the acoustic waveform. In some variations, the acoustic waveform and the reflected acoustic waveform may each be configured to be immune to coupling with an external noise waveform. In some variations, the external noise waveform may include one or more of a sound waveform other than those produced by the subject, an external ultrasound waveform, and an external electromagnetic waveform. In some variations, the acoustic waveform may be transmitted continuously. In some variations, the reflected acoustic waveform may be measured continuously.

In some variations, the acoustic waveform may be transmitted from between about 5 cm and about 15 cm away from the skin of the subject. In some variations, the reflected acoustic waveform may be measured from between about 5 cm and about 15 cm away from the skin of the subject. In some variations, the acoustic waveform may include a frequency of between about 40 kHz and about 300 kHz. In some variations, the acoustic waveform may include a field of view of between about 30 degrees and about 120 degrees. In some variations, the acoustic waveform may include a sound pressure level between about 90 dB and about 135 dB at about 30 cm.

In some variations, the skin of the subject may include a skin of one or more of a neck, a jaw, and a chin. In some variations, the skin of the neck may include a skin covering one or more of a larynx, a circloid cartilage, a thyroid cartilage, a thyroid gland, a trachea, and a lymph node.

In some variations, processing the measured reflected waveform may include processing a scattered acoustic waveform. In some variations, transmitting the acoustic waveform and measuring the reflected acoustic waveform may include using at least one transducer. In some variations, the methods may further include calibrating the at least one transducer based on a distance between the transducer and the skin of the subject. In some variations, the techniques described herein relate to a method, wherein the at least one transducer includes a sensitivity of between about 0.01 V/Pa and about 100 V/Pa.

In some variations, processing the measured reflected acoustic waveform may include estimating a change in one or more of a phase and a frequency of the measured reflected acoustic waveform. In some variations, processing the measured reflected acoustic waveform may include applying a quadrature demodulation to the measured reflected acoustic waveform. In some variations, processing the measured reflected acoustic waveform may include inputting the measured reflected acoustic waveform to a phase lock loop circuit. In some variations, processing the measured reflected acoustic waveform may include applying a Hilbert transformation to the measured reflected acoustic waveform.

In some variations, the methods may include transcribing the vocal data to text data. In some variations, the methods may include equalizing the vocal data. In some variations, the methods may include inputting the vocal data to a machine learning model and generating a response using the machine learning model. In some variations, the methods may include analyzing the vocal data using a natural language model to generate a response. In some variations, the methods may include communicating the response of the natural language model to the subject. In some variations, the methods including transmitting the vocal data using a communication device to one or more of the subject, another subject, and a computing device.

In some variations, the methods may include releasably coupling a device to a subject, the device including a support configured to releasably couple to the subject, at least one transducer coupled to the support, and a processor and memory coupled to the support. In some variations, the at least one transducer is between about 5 cm and about 15 cm away from the skin of the subject. In some variations, the at least one transducer is configured to transmit the acoustic waveform at a frequency of between about 40 kHz and about 300 kHz. In some variations, the at least one transducer is configured to transmit the acoustic waveform in a field of view of between about 30 degrees and about 120 degrees. In some variations, the at least one transducer is configured to measure a scattered acoustic waveform. In some variations, releasably coupling the device includes coupling the device to one or more of a neck, a shoulder, a back, and a chest of a subject.

In some variations, the methods may include analyzing the vocal data using a natural language model to generate a response. In some variations, the techniques described herein relate to a method, further including communicating the response to the subject.

Also described here are sensors and systems. In some variations, a sensor may comprise a device for generating vocal data, including: at least one transducer configured to transmit an acoustic waveform through air to a skin of a subject and measure a reflected acoustic waveform; a support coupled to the at least one transducer, the support configured to be releasably coupled to the subject; and a processor and a memory coupled to the support, the processor configured to: transmit the acoustic waveform to the skin of the subject; measure the reflected acoustic waveform reflected by the skin; and generate vocal data of the subject based on a change in the reflected acoustic waveform relative to the acoustic waveform.

In some variations, the reflected acoustic waveform may include the acoustic waveform coupled with a voice signal corresponding to a vibration in the skin. In some variations, reflected acoustic waveform may include a change in one or more of a phase and a frequency relative to the acoustic waveform.

In some variations, the at least one transducer may be configured to transmit the acoustic waveform such that the acoustic waveform and the reflected acoustic waveform are each may be configured to be immune to coupling with an external noise waveform. In some variations, the external noise waveform may include one or more of a sound waveform other than those produced by the subject, an external ultrasound waveform, and an external electromagnetic waveform.

In some variations, the at least one transducer may be configured to transmit acoustic waveform continuously. In some variations, the at least one transducer may be configured to measure the reflected acoustic waveform continuously.

In some variations, the support includes a concave shape configured to releasably couple to one or more of a neck, a shoulder, a back, and a chest of a subject. In some variations, the transducer is between about 5 cm and about 15 cm away from the skin of the subject.

In some variations, the at least one transducer includes a transmit configuration configured to transmit the acoustic waveform and a receive configuration configured to measure the reflected acoustic waveform.

In some variations, a first transducer configured to transmit and a second transducer configured to receive are disposed on opposite lateral sides of a subject. In some variations, the at least one transducer includes an array of receivers configured to measure the reflected acoustic waveform. In some variations, the at least one transducer is configured to transmit the acoustic waveform at a frequency of between about 40 kHz and about 300 kHz.

In some variations, the at least one transducer is configured to transmit the acoustic waveform at a sound pressure level between about 90 dB and about 135 dB at about 30 cm. In some variations, the at least one transducer is configured to transmit the acoustic waveform in a field of view of between about 30 degrees and about 120 degrees.

In some variations, the at least one transducer includes a sensitivity of between about 0.01 V/Pa and about 100 V/Pa. In some variations, the at least one transducer is configured to receive the reflected acoustic waveform including a frequency between about 40 kHz and about 300 kHz.

In some variations, the at least one transducer is configured to measure a scattered acoustic waveform. In some variations, the processor is configured to generate vocal data of the subject based on an estimated change in a scattered acoustic waveform relative to the acoustic waveform. In some variations the processor is configured to estimate a change in one or more of phase and frequency of the reflected acoustic waveform.

In some variations, the processor is further configured to apply a machine learning model to the vocal data and generate a response. In some variations, the processor is further configured to analyze the vocal data using a natural language model to generate a response. In some variations, the processor is further configured to communicate the response of the natural language model to the subject.

Additional methods for generating voice data are also described herein. For example, a method of generating vocal data may include continuously transmitting an acoustic waveform to a skin of a subject, continuously measuring the acoustic waveform coupled with a voice signal, and estimating a modulation of the measured acoustic waveform to generate vocal data of the subject.

In some variations, the acoustic waveform coupled with the voice signal may include a change in one or more of a phase and a frequency relative to the transmitted acoustic waveform. In some variations, the voice signal may correspond to a vibration in the skin of the subject. In some variations, the acoustic waveform coupled with the voice signal may be reflected by the skin of the subject.

In some variations, the acoustic waveform and the acoustic waveform coupled with the voice signal may each be configured to be immune to coupling with an external noise waveform. In some variations, the external noise waveform may include one or more of a sound waveform other than those produced by the subject, an external ultrasound waveform, and an external electromagnetic waveform.

In some variations, the acoustic waveform may be transmitted from between about 5 cm and about 15 cm away from the skin of the subject. In some variations, the acoustic waveform may be measured from between about 5 cm and about 15 cm away from the skin of the subject. In some variations, the acoustic waveform may include a frequency of between about 40 kHz and about 300 kHz. In some variations, the acoustic waveform may include a field of view of between about 30 degrees and about 120 degrees. In some variations, the acoustic waveform may include a sound pressure level between about 90 dB and about 135 dB at about 30 cm.

In some variations, the skin of the subject may include a skin of one or more of a neck, a jaw, and a chin. In some variations, the skin of the neck may include a skin covering one or more of a larynx, a circloid cartilage, a thyroid cartilage, a thyroid gland, a trachea, and a lymph node. In some variations, measuring the acoustic waveform may include measuring a scattered acoustic waveform.

In some variations, estimating the modulation of the measured acoustic waveform may include estimating a change in one or more of a phase and a frequency of the measured acoustic waveform. In some variations, estimating the modulation of the measured acoustic waveform may include applying a quadrature demodulation to the measured acoustic waveform. In some variations, estimating the modulation of the measured acoustic waveform may include inputting the measured acoustic waveform to a phase lock loop circuit. In some variations, estimating the modulation of the measured acoustic waveform may include applying a Hilbert transformation to the measured acoustic waveform.

In some variations, the method may further include transcribing the vocal data to text data. In some variations, the method may further include inputting the vocal data to a machine learning model and generating a response using the machine learning model. In some variations, the method may further include analyzing the vocal data using a natural language model to generate a response. In some variations, the method may further include communicating the response of the natural language model to the subject. In some variations, the method may further include transmitting the vocal data using a communication device to one or more of the subject, another subject, and a computing device.

Additional sensors and systems for generating voice data are also described herein. For example, a device for generating vocal data may include at least one transducer configured to continuously transmit an acoustic waveform to a skin of a subject and continuously measure the acoustic waveform coupled with a voice signal, a support coupled to the at least one transducer, the support configured to be releasably coupled to the subject; and a processor and a memory coupled to the support, the processor may be configured to continuously transmit an acoustic waveform to the skin of the subject, continuously measure the acoustic waveform coupled with the voice signal, and estimating a modulation of the measured acoustic waveform to generate vocal data of the subject.

In some variations, the at least one transducer may be configured to transmit the acoustic waveform such that the acoustic waveform couples with a voice signal corresponding to a vibration in the skin. In some variations, the acoustic waveform coupled with a voice signal may include a change in one or more of a phase and a frequency relative to the acoustic waveform.

In some variations, the at least one transducer may be configured to transmit the acoustic waveform configured to be immune to coupling with an external noise waveform. In some variations, the external noise waveform may include one or more of a sound waveform other than those produced by the subject, an external ultrasound waveform, and an external electromagnetic waveform.

In some variations, the at least one transducer may be configured to transmit the acoustic waveform continuously. In some variations, the at least one transducer may be configured to measure the acoustic waveform coupled with the voice signal continuously.

In some variations, the support may include a concave shape configured to releasably couple to one or more of a neck, a shoulder, a back, and a chest of a subject. In some variations, the transducer may be between about 5 cm and about 15 cm away from the skin of the subject.

In some variations, wherein the at least one transducer may include a transmit configuration configured to transmit the acoustic waveform and a receive configuration configured to measure the acoustic waveform coupled with the voice signal. In some variations, a first transducer configured to transmit and a second transducer configured to receive may be disposed on opposite lateral sides of a subject.

In some variations, the at least one transducer may include an array of receivers configured to measure the acoustic waveform coupled with the voice signal.

In some variations, the at least one transducer may be configured to transmit the acoustic waveform at a frequency of between about 40 kHz and about 300 kHz. In some variations, the at least one transducer is configured to transmit the acoustic waveform at a sound pressure level between about 90 dB and about 135 dB at about 30 cm. In some variations, the at least one transducer may be configured to transmit the acoustic waveform in a field of view of between about 30 degrees and about 120 degrees.

In some variations, the at least one transducer may include a sensitivity of between about 0.01 V/Pa and about 100 V/Pa. In some variations, the at least one transducer may be configured to receive the acoustic waveform coupled with the voice signal comprising a frequency between about 40 kHz and about 300 kHz.

In some variations, the at least one transducer may be configured to measure a scattered acoustic waveform. In some variations, the processor may be configured to generate vocal data of the subject based on an estimated change in a scattered acoustic waveform relative to the acoustic waveform. In some variations, the processor may be configured to estimate a change in one or more of phase and frequency of the acoustic waveform coupled with the voice signal.

In some variations, the processor may be further configured to apply a machine learning model to the vocal data and generate a response. In some variations, the processor may be further configured to analyze the vocal data using a natural language model to generate a response. In some variations, the processor may be further configured to communicate the response of the natural language model to the subject.

Additional devices for generating vocal data are described herein. For example, a device for generating vocal data may comprise a transducer configured to transmit an acoustic waveform through air to a skin of a subject, one or more MEMS microphones configured to measure a reflected acoustic waveform, a support coupled to the transducer and the one or more MEMS microphones, where the support is configured to be releasably coupled to the subject, and a processor and a memory coupled to the support. The processor may be configured to transmit the acoustic waveform to the skin of the subject, measure the reflected acoustic waveform reflected by the skin, and generate vocal data of the subject based on a change in the reflected acoustic waveform relative to the acoustic waveform.

In some variations, the reflected acoustic waveform may comprise the acoustic waveform coupled with a voice signal corresponding to a vibration in the skin. In some variations, the reflected acoustic waveform may comprise a change in one or more of a phase and a frequency relative to the acoustic waveform. In some variations, the one or more MEMS microphones may be configured to measure the reflected acoustic waveform continuously. In some variations, the transducer may be between about 5 cm and about 15 cm away from the skin of the subject.

In some variations, the one or more MEMS microphones may comprise a plurality of MEMS microphones configured to measure the reflected acoustic waveform. In some variations, the processor may be further configured to select a MEMS microphone of the plurality of MEMS microphones to measure the reflected acoustic waveform based on a signal to noise ratio of the reflected acoustic waveform measured by the MEMS microphone compared to other MEMS microphones of the plurality of MEMS microphones.

In some variations, the one or more MEMS microphones may be configured to receive the reflected acoustic waveform with a field of view between about 90 degrees and about 180 degrees. In some variations, the one or more MEMS microphones may be configured to measure a scattered acoustic waveform.

### BRIEF DESCRIPTION OF THE DRAWINGS

FIG. 1 is a schematic block diagram of an illustrative variation of a system for generating vocal data.

FIG. 2 is a schematic diagram of an illustrative variation of a sensor measuring a reflected signal waveform.

FIGS. 3A, 3B, and 3C are schematic side views of illustrative variations of a sensor releasably coupled to a subject.

FIGS. 4A and 4B are schematic side views of illustrative variations of a sensor releasably coupled to a subject.

FIG. 5 is a perspective view of an illustrative variation of a sensor.

FIG. 6A is a schematic side view of a transducer array transmitting to a subject. FIG. 6B is a schematic side view of a sensor releasably coupled to a subject.

FIG. 7 is a flowchart describing an illustrative variation of a method of generating vocal data from a subject.

FIG. 8 is a flowchart describing an illustrative variation of a method of processing a signal waveform.

FIG. 9 is a flowchart describing an illustrative variation of another method of processing a signal waveform.

FIG. 10 is a flowchart describing an illustrative variation of yet another method of processing a signal waveform.

FIGS. 11A, 11B, and 11C are a perspective view, a top view, and a side view respectively of an illustrative variation of a wearable sensor.

### DETAILED DESCRIPTION

Described here are systems, devices, and methods for generating vocal data from a subject. These systems, devices, and methods may transmit, measure, and process acoustic waveforms to generate vocal data. For example, the systems, methods, and devices may generate vocal data used to allow the subject to communicate with a natural language model, other device, or another person. Generally, a device for generating vocal data may include one or more transducers, a support, and a processor and memory. The device may further be comfortably worn (e.g., coupled to) on a portion of a subject's body for extended periods of time. The device may have a form factor such as an open necklace or collar worn around, for example, the shoulders. The device may monitor the speech vibrations in a neck of the wearer by bouncing an ultrasonic wave off of the neck that's reflected back to the device. The reflected ultrasound wave may incorporate (e.g., collect, carry) the speech vibrations from back to the device. The device may then generate vocal data of the wearer by extracting the vibrations of the neck from the ultrasound wave as described in more detail herein. The systems, devices and methods described herein may improve voice audio recording by: facilitating continuous generation of vocal data in a non-contact manner; reducing the presence of external noise in generated vocal data; enabling detection of low volume voices in high noise environments; minimizing calibration of the device to increase ease-of-use for the subject; increasing robustness and reliability of measuring to reduce need for additional alignment or calibration; improving comfort and reducing intrusion of the device to promote adoption and use; and delivering a lightweight and compact form factor which facilitates extended use and wearability.

Conventional devices and methods of recording vocal data suffer from numerous deficiencies that are addressed by the systems, device, and methods described herein. For example, conventional contact throat microphones provide measurement of vocal data by maintaining physical contact with sensitive areas of the subject such as the throat and neck and monitoring the vibrations of a subject via a contact sensor. Due to the intrusiveness and discomfort experienced by users of such devices, these conventional solutions are not suitable for extended use and are typically reserved for extremely noise environments where voice communication supersedes comfort (e.g., military operations, motorcycle riding, industrial settings). Other conventional techniques include laser vibrometry which measures the vibrations from a surface by splitting a beam of laser light and sending one beam to a vibrating surface and the other to a sensor. The first light beam bounces off of the vibrating surface and returns to the sensor with information about the vibration of the surface. Such techniques require analog processing, which may limit the ability to simulate data, are susceptible to interference (e.g., speckle) upon reflection, monitor a limited field of view (FOV) resulting in a loss of data or additional calibration, and may require calibration and proper environmental light conditions for the sensor to function properly.

Generally, the systems, devices, and methods described herein may facilitate generation of vocal data from a subject in a non-contact manner that is substantially immune to non-voice sound (e.g., external noise). The systems, devices and methods generally utilize a device (e.g., sensor) configured to detect vibrations in a subject. In some variations, the sensor may include a transducer configured to detect vibrations by measuring a modulated signal waveform. The transducer may be configured to transmit signal waveform(s) (e.g., acoustic waveforms) through a gaseous medium (e.g., air) and measure the signal waveform(s) reflected by the subject. In some variations, the transducer may be configured to measure signal waveforms(s) limited to a predetermined frequency range (e.g., ultrasound) to reduce undesirable noise. In some variations, a receiver (e.g., ultrasound receiver, micro-electro-mechanical system (MEMS) microphone) may be configured to measure the signal waveform(s) reflected by the subject. The sensor may be coupled to a support where the support may be releasably coupled to a predetermined portion (e.g., neck, shoulders) of the subject. The sensor may be held in a fixed position relative to the subject (e.g., even when the subject moves).

Furthermore, the systems, devices, and methods described herein may facilitate generating vocal data from a subject in manner more comfortable and less intrusive than existing systems and devices. The systems and devices described herein generally facilitate the generation of vocal data by transmitting a signal waveform and measuring a reflected signal waveform. In some variations, the sensor may be configured to measure the reflected signal waveform of subject without making physical contact with the area of the subject reflecting the reflected signal waveform. For example, the transducer may be configured to measure an acoustic waveform reflect by the skin of the neck at a distance of between about 5 cm and about 15 cm away from the skin of the neck. This may improve subject comfort, thereby increasing adoption and use.

Generally, the vocal data generating systems described here may comprise a sensor configured to measure reflected signal waveforms (e.g., acoustic waveforms) and generate vocal data of a subject. In some variations, the vocal data may be generated by the sensor itself, while in other variations, the sensor may transmit the reflected signal waveforms to one or more external devices (e.g., a computing device, a dock, and/or a database) where the reflected signal waveforms may be processed and/or the vocal data may be generated. In some variations, the sensor may generate the vocal data and may then transmit the vocal data to a computing device, a dock, and/or a database for processing and/or analysis.

Generally, the methods described herein may generate vocal data using a sensor including a transducer (e.g., ultrasound transducer). For example, the method may include transmitting an acoustic waveform, measuring acoustic waveforms reflected by the subject, for example using a MEMS microphone, and processing the reflected acoustic waveforms to generate vocal data. Furthermore, processing reflected acoustic waveforms (e.g., ultrasound signals) may comprise processing acoustic waveforms reflected by the subject (e.g., scattered acoustic waveforms). The generation of vocal data may be performed at predetermined intervals or continuously. The results of the data processing may be output to one or more of an output device of the sensor, a computing device, a dock, a network, a server, a database, a natural language model, one or more designated contacts or combinations thereof, and the like. A voice recording may be one or more the spoken words of a subject, other audible sounds, and inaudible expressive noises that register vibrations in the subject.

Additionally or alternatively, the vocal data generating devices, systems, and methods described herein may be incorporated into a variety of environments and applications, including vehicles such as cars, planes, helicopters, trucks, industrial vehicles (e.g., forklifts, cranes, theme park rides), as well as stadium seating, concert venues, musician setups, stage performances, and/or office settings. These implementations are provided by way of example and are not intended to be limiting. The devices, systems, and methods described herein may be adapted for use in other suitable applications where vocal data generation is beneficial.

### I. Systems And Devices

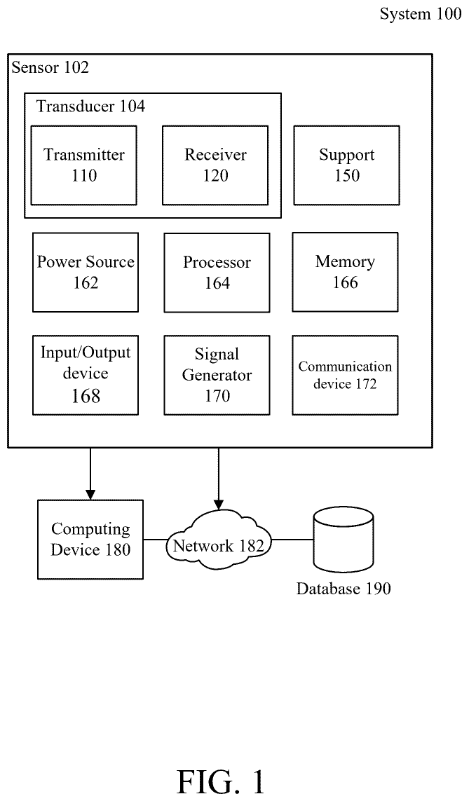

A vocal data generation system may include one or more of the following components necessary to measure and/or generate vocal data (e.g., audio vocal data) using the devices as described herein. FIG. 1 is a block diagram of a variation of a vocal data generation system (100).

As shown there, the system (100) may comprise a sensor (102), and optionally one or more of a computing device (180), a network (182), and a database (190). The system (100) may comprise a sensor (102) configured to removably attach to a subject (not shown). The sensor (102) may be configured to receive signal waveforms transmitted to and reflected by the subject. As described in more detail herein, the sensor (102) may comprise a transducer (104) comprising a transmitter (110) configured to transmit one or more signal waveforms and a receiver (120) configured to measure one or more reflected signal waveforms, a signal generator (170) configured to generate the signal waveforms, an input/output device (168) configured to receive input and generate output to a user, a processor (164) and a memory (166) configured to control the sensor (102), a communication device (172) configured to establish a communication channel to communicate with other components in the system (100), a power source (162) configured to power the sensor (102), and a support (152) configured to releasably attach to the sensor (e.g., the transducer) to the subject (e.g., shoulders of the subject). The transducer (104) may comprise one or more transducers such as a transducer array comprising a plurality of transducers. In some variations, the transmitter (110) and the receiver (120) may be the same component. In other variations, the transmitter (110) and the receiver (120) may be separate components.

In some variations, the sensor (102) may be operatively coupled to a computing device (180) through one or more wired or wireless communication channels. The computing device (180) may be operatively coupled one or more networks (182), databases (190), servers, and the like. The network (182) may comprise one or more databases (190) and computing devices (180). In some variations, the sensor (102) may be coupled directly (e.g., physically) to any of a dock (not shown), the computing device (180), the network (182), and the database (190). In some variations, the sensor (102) may be coupled to the dock for storage, to recharge, to transfer data, combinations thereof, and the like.

In some variations, the measured reflected signal waveform (e.g. reflected acoustic waveform) data may be processed on the sensor (102) (e.g., processed by one or more of the processor (164) and memory (166)) and/or the processing of the measured waveform data may be distributed throughout the system (100) (e.g., processed by one or more of the computing device (180), network (182)). In some variations, the reflected signal waveform data processing may comprise one or more of filtering data (e.g., reducing noise, averaging waveforms), converting data from analog to digital, demodulating the data, and generating vocal data based on the reflected signal waveform.

### Sensor

Generally, the sensors described here may be configured to measure vocal data on a predetermined (e.g., continuous) basis. In some variations, the sensor may transmit one or more signal waveforms and measure the signal waveforms reflected by tissue. The measured reflected signal waveforms (e.g., reflected acoustic waveforms, modulated acoustic waveforms) may be processed to generate vocal data corresponding to the voice of the subject. In some variations, the sensor may be controlled by one or more computing devices. For example, input commands may be input to the computing device and transmitted to the sensor to operatively control the sensor. In some variations, the sensors described here may be configured to perform a subset of the transmission, measurement, processing, and generation steps described in more detail herein. For example, the reflected signal waveforms measured by the sensor may be transmitted to a separate computing device for vocal data generation, analysis, communication, etc.

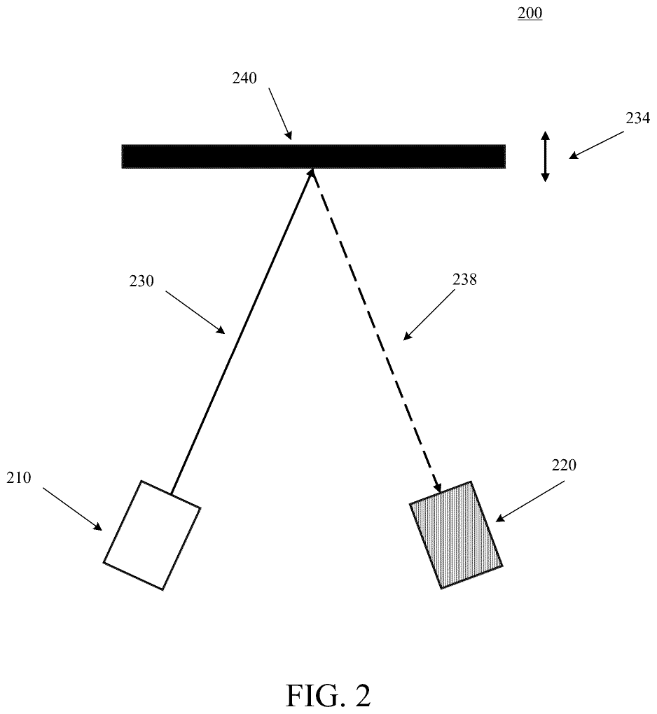

Generally, the sensors described herein may generate vocal data of a subject by measuring a reflected signal waveform. For example, FIG. 2 is a schematic diagram (200) of an illustrative variation of a sensor including respective transducers (210, 220) transmitting a signal waveform (230) (e.g., acoustic waveform) and receiving a reflected signal waveform (238) (e.g., reflected acoustic waveform), a surface of a subject (240), and a voice signal (234). The surface of the subject (240) may correspond to a skin of the subject that may vibrate due to internal vibrations (e.g., from a set of vocal cords). The voice signal (234) may correspond to the vibrations in the skin of the subject caused by the subject speaking (e.g., generating vocal sounds). In some variations, a first transducer (210) may be configured to transmit an acoustic waveform (230) to the vibrating surface of the subject (240) (e.g., skin of the neck), and a second transducer (220) (e.g., receiver) may be configured to measure the reflected acoustic waveform (238). In some variations, the signal waveform (230) may couple to the voice signal (234) such that the reflected signal waveform (238) comprises both the signal waveform (230) and the voice signal (234). In some variations, a difference in impedance between the vibrating surface of the subject (240) and a gaseous medium (e.g., air) may cause the signal waveform to reflect.

Generally, the sensors described herein may measure a reflected signal waveform (238) comprising the voice signal (234) of the subject. For example, the reflected signal waveform (238) may comprise the signal waveform (230) coupled with the voice signal (234). For example, the surface of the subject (240) vibrating based on the voice signal (234) may contact and thereafter modulate the signal waveform (230) such that the reflected signal waveform (238) includes the voice signal (234). For example, the surface of the subject (e.g., skin of the neck) may define a normal axis perpendicular to the surface. Vocalization by the subject may then cause the surface to move (e.g., vibrate) partially along the normal axis. A transducer (210, 220) may be configured to transmit a signal waveform (230) such that movement of the skin along the normal axis modulates the signal waveform (230) upon contact, thereby returning the reflected signal waveform (238) comprising the voice signal (234) and the signal waveform (230). The reflected signal waveform (238) may comprise a change in one or more of phase and frequency relative to the signal waveform (230). In some variations, the voice signal may comprise the audible voice of the subject. The audible voice data may be measured by the transducer (220) along with the modulated signal waveform (238).

In some variations, the transducer (210, 220) may be configured to transmit an acoustic waveform (230) to the skin of the subject. The vibrating surface (240) of the subject may comprise the skin of on one or more of the neck, jaw, and chin of the subject. More specifically, the skin of the neck may include the skin covering one or more of the larynx, the circloid cartilage, the thyroid cartilage, the thyroid gland, the trachea, the lymph nodes, and other similar structures of the neck vibrated by the subject when speaking. In some variations, the signal waveform (230) and the reflected signal waveform (238) may be transmitted such that neither is substantially modified by other waveforms in the environment (e.g., noise). In some variations, the signal waveform (230) and reflected signal waveform (238) may be configured to be substantially immune to coupling with external noise waveform(s). As discussed herein, the reflected signal waveform (238) includes the transmitted signal waveform coupled to the voice signal (234) and is further substantially absent coupling with other sources of interference (e.g., noise). External noise waveforms may include one or more sound waveforms other than those produced by the subject, an external ultrasound waveform, and an external electromagnetic waveform (e.g., visible light). For example, the signal waveform (230) and reflected signal waveform (238) may comprise an ultrasound frequency configured to minimize coupling with ambient sound waves in the environment. In this manner, the generated vocal data is substantially absent interference from external noise sources (e.g., other sounds) such as wind noise, other speakers, environmental sounds, and the like. Thus, rather than requiring post-processing (e.g., noise-cancelation) of the generated vocal data to remove non-speaker noise, the reflected signal waveform (238) coupled with the voice signal (234) may be substantially immune from non-speaker voice waveforms. In some variations, the transducer (210, 220) may be configured to measure the acoustic waveform scattered by the skin of the subject (240), thereby increasing the robustness of the device and minimizing one or more calibration and alignment of the sensor (e.g., transducers (210, 220)). In some variations, the transducer (210, 220) may be configured to measure a scattered acoustic waveform substantially absent modulation by other signals in the environment, thereby increasing a signal-to-noise ratio of the vocal data.

### Transducer

Generally, the transducer may be configured to transmit and/or receive acoustic waveforms to collect vocal data of the subject. In some variations, the transducer for generating and/or transmitting an acoustic signal (e.g., an ultrasound signal) may comprise, for example, one or more of a piezoelectric transducer, a lead zirconate titanate (PZT) transducer, a polymer thick film (PTF) transducer, a polyvinylidene fluoride (PVDF) transducer, a capacitive micromachined ultrasound transducer (CMUT), a piezoelectric micromachined ultrasound transducer (PMUT), a photoacoustic transducer, a transducer based on single crystal materials (e.g., LiNbO₃(LN), Pb(Mg_{1/3}Nb_{2/3})—PbTiO₃ (PMN-PT), and Pb(In_{1/2}Nb_{1/2})—Pb(Mg_{1/3}Nb_{2/3})—PbTiO₃ (PIN-PMN-PT)), and/or any suitable component for generating and/or transmitting an acoustic signal. A transducer configured to receive acoustic waveforms may comprise one or more of a piezoelectric transducer, a PZT transducer, a PTF transducer, a PVDF transducer, a CMUT, a PMUT, a photoacoustic transducer, a transducer based on single crystal materials, as well as a Micro-Electro-Mechanical Systems (MEMS) microphone configured to receive ultrasonic and/or audible acoustic signals. In some variations, the sensor may comprise an analog MEMS microphone. An analog MEMS microphone may be advantageous by providing direct access to a signal from a membrane of the MEMS microphone.

In some variations, the transducer (210, 220) may be configured to transmit an ultrasonic acoustic waveform to the skin of the subject. The transducer (210, 220) may be configured to transmit the ultrasonic acoustic waveform (230) at a frequency that provides sufficient bandwidth to include the voice signal (234) when reflected. In some variations, the transducer (210, 220) may be configured to transmit an acoustic waveform (230) comprising a single predetermined frequency. For example, the transducer may be configured to transmit the acoustic waveform (e.g., ultrasound pressure wave) at a frequency of between of about 40kHz to about 300 kHz, about 40 kHz to about 200 kHz, about 40 kHz to about 100 kHz, about 100 kHz to about 300 kHz, about 100 kHz to about 200 kHz, and about 150 kHz to about 250 kHz, including all subranges and values therebetween. For example, the transducer may be configured to transmit the acoustic waveform at a single frequency of about 40 kHz, about 80 kHz, about 100 kHz, about 150 kHz, about 200 kHz, about 250 kHz or about 300 kHz. In some variations, the transducer (210, 220) may be configured to change the frequency of the acoustic waveform (230) while measuring the reflected acoustic waveform (238), thereby increasing the robustness of the device and minimizing one or more of calibration and alignment of the sensor.

In some variations, the transducer (210, 220) may be configured to transmit an acoustic waveform (230) such that the acoustic waveform (230) reflects off of a vibrating surface of the subject (240). For example, the transducer (210) may be configured to transmit at a sound pressure level between about 90 dB and about 135 dB at about 30 cm, between about 90 dB and about 120 dB at about 30 cm, between about 105 dB and about 135 dB at about 30 cm, and between about 105 dB and about 120 dB at about 30 cm. For example, the transducer (210, 220) may be configured to transmit the acoustic waveform (230) at a sound pressure level of about 90 dB at about 30 cm, about 100 dB at about 30 cm, about 110 dB at about 30 cm, about 115 dB at about 30 cm, about 125 dB at about 30 cm, and about 135 dB at about 30 cm, including all subranges and values therebetween. In some variations, the transducer (210, 220) may be configured to transmit the acoustic waveform (230) within a field of view configured to target the vibrating surface of the subject (240) and reflect to the transducer (210, 220). For example, the transducer (210, 220) may be configured to transmit the acoustic waveform (230) of between about 30 degrees and about 120 degrees. For example, the transducer (210, 220) may be configured to transmit the acoustic waveform with a field of view of about 30 degrees, about 50 degrees, about 70 degrees, about 90 degrees, and about 120 degrees.

The transducer (210, 220) may also be configured to measure the reflected acoustic waveform (238). In some variations, the transducer may be configured to receive a limited range of frequencies to avoid undesired noise. For example, the transducer may be configured to receive reflected acoustic waveforms with a frequency of between about 40kHz to about 300 kHz, between about 40 kHz to about 200 kHz, between about 40 kHz to about 100 kHz, between about 100 kHz to about 300 kHz, between about 100 kHz to about 200 kHz, and between about 150 kHz to about 250 kHz, including all subranges and values therebetween. In some variations the transducer may have a sensitivity configured to measure the reflected acoustic waveform. For example, the transducer may have a sensitivity of between about 0.01 V/Pa and about 100 V/Pa, between about 0.1 V/pa and about 100 V/Pa, between about 0.01 V/Pa and about 10 V/Pa, and between about 0.1 V/Pa and about 10 V/Pa, including all subranges and values therebetween.

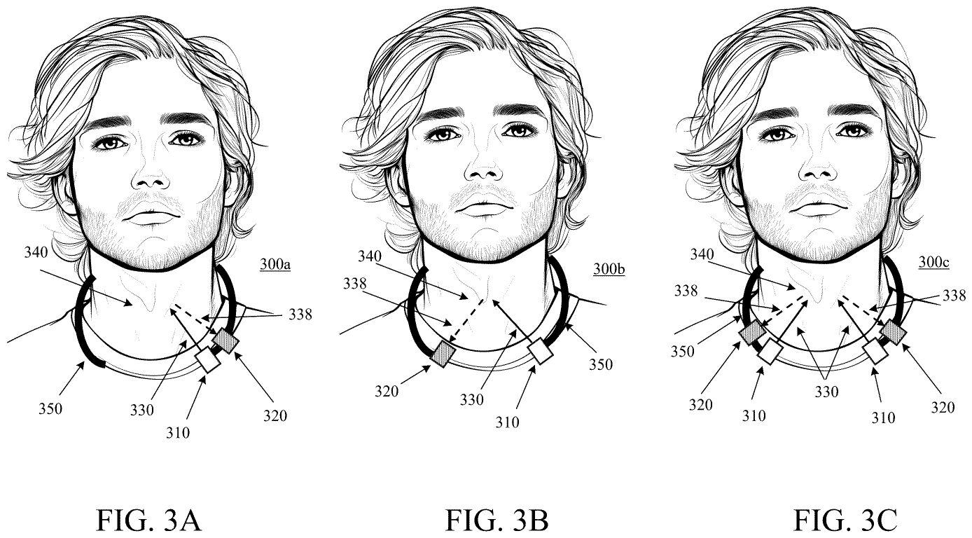

Generally, the sensors described herein may be disposed around a subject and generate vocal data without physically contacting the vibrating surface of the subject (e.g., the neck). FIG. 3A is a schematic side view of an illustrative variation of a sensor (300a) releasably coupled to a subject. The sensor (300a) may comprise one or more transducers (310, 320) configured to transmit an acoustic waveform (330) and measure a reflected acoustic waveform (338) and a support (350) configured to releasably couple the sensor (300a) to the subject. As shown in FIG. 3A, one or more transducers may include a transducer in a transmitter configuration (310) configured to transmit the acoustic waveform (330) and a transducer in a receive configuration (320) configured to measure the reflected acoustic waveform (338). In some variations, one or more transducers (310, 320) may be configured to measure the surface of the subject (340) without physically contacting the surface (340). For example, the transducer (310, 320) may be between about 5 cm and about 15 cm away from the skin of the neck. This configuration of the transducers relative to the subject may improve the comfort and the compactness of the sensor (300a). For example, the sensor (300a) may be disposed on less sensitive and/or intrusive portions of the body such as the shoulders, and may also be more aesthetically appealing than a contact microphone placed on the neck.

In some variations, one or more transducers may be placed apart (e.g., spaced apart, separated) from one another to improve the measurement of the reflected acoustic waveform based on the dimensions of the subject (e.g., user, wearer). FIGS. 3B and 3C are schematic side views of illustrative variations of a sensor releasably coupled to a subject. The sensor (300b, 300c) may comprise one or more transducers (310, 320) configured to transmit an acoustic waveform (330) and measure a reflected acoustic waveform (338) and a support (350) configured to releasably couple to the subject. As shown in FIG. 3B, the sensor (300b) may comprise a first transducer (310) configured to transmit and a second transducer (320) (e.g., MEMS microphone) configured to receive disposed on opposite lateral sides of the subject. In some variations, a transducer (310) may be configured to transmit an acoustic waveform (330) such that the acoustic waveform (330) reaches the vibrating surface of the subject (340) and is diffused (e.g., scattered) in multiple directions by the surface of the subject (340). Thus, the reflected acoustic waveform (338) may comprise a scatted acoustic waveform, and a transducer (320) may be configured to measure the scattered acoustic waveform.

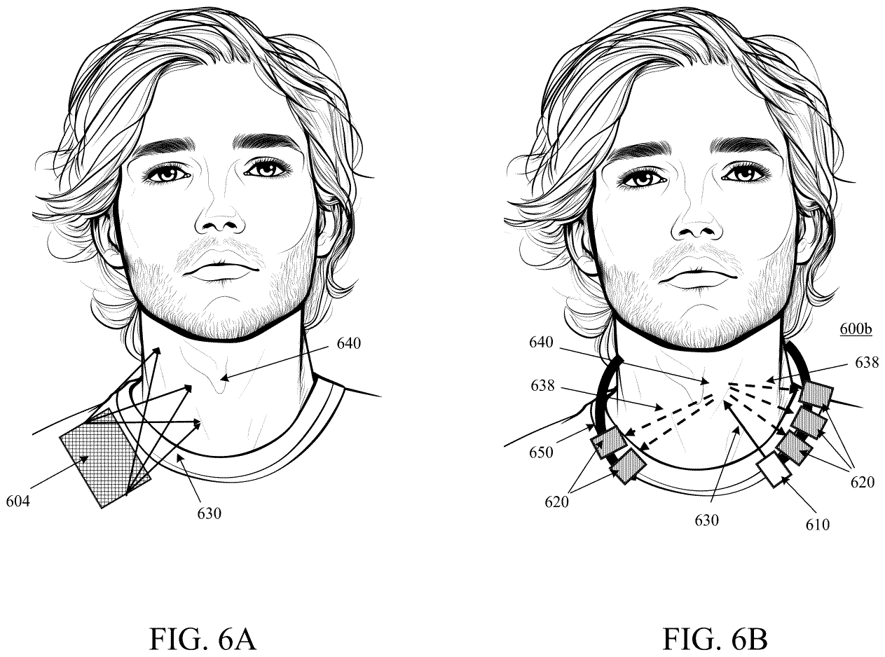

In some variations, the sensor may include a plurality of transducers for more robust vocal data generation. FIG. 6A is a schematic side view of a transducer array (604) comprising a plurality of transducers transmitting to a subject. In some variations, the transducer array may include a plurality of separately driven transducer elements. The transducer array may comprise any suitable ultrasound transducer array, including one or more of a linear array, a curved array, a phased array, and the like. In some variations, a transducer of a transducer array may include one or more of a 1D transducer, a 1.5D transducer, a 2D transducer, a 3D transducer, and the like. The transducer array may be configured to transmit an acoustic waveform (630) to the vibrating surface of the subject (640). In some variations, as shown in FIG. 6A, the transducer array (604) may be configured to target the acoustic waveform (630) to one or more locations on the vibrating surface of the subject (640) (e.g., skin of either side of the neck, skin under the chin). Additionally or alternatively, the transducer array (604) may be configured to transmit the acoustic waveform (630) with a field of view wide enough to encompass one or more locations (e.g., neck, a jaw, a chin, a larynx, a circloid cartilage, a thyroid cartilage, a thyroid gland, a trachea, and a lymph node) on the vibrating surface of the subject (640). For example, the transducer array (604) may be configured to transmit the acoustic waveform (630) with a field of view of between about 30 degrees and about 120 degrees and target one or more locations on the vibrating surface of the subject (640) simultaneously.

In some variations, the sensor may include a plurality of transducers configured as receivers to improve the robustness of the vocal data generation system and decrease undesired noise in the generated vocal data. As shown in FIG. 6B, a plurality of transducers configured as receivers (620) may be coupled to a support (650) and configured to measure the reflected acoustic waveform (638). For example, the plurality of transducers configured as receivers (620) may be configured to receive a reflected acoustic waveform (638) (e.g., scattered waveform) transmitted by one or more transducers configured as transmitters (610) and reflected off the vibrating surface of the subject (640). In some variations, a single transducer may be configured as a transmitter, and a plurality of receivers (e.g., MEMS microphones) may be configured as receivers. For instance, the sensor may comprise a single transmitter when one or more of the plurality of receivers has a larger field of view, such as when the receiver is a MEMS microphone. The sensor (600b) may additionally include a processor and a memory (not shown) configured to process a plurality of reflected acoustic waveforms (638) measured by the plurality of transducers configured as receivers (620). This configuration may reduce to reduce noise in the generated vocal data by linearly increasing the strength of the reflected acoustic waveforms based on the number of receivers (620). For example, a signal-to-noise ratio of the measured reflected acoustic waveform (638) may improve as function of n/sqrt(n), where n is the number of transducers configured as receivers.

In some variations, the one or more transducer configured as receivers may be MEMS microphones. For example, a sensor may comprise a plurality of MEMS microphones configured to measure the reflected acoustic waveform. In some variations, A MEMS microphone may be configured to measure an ultrasound waveform. A MEMS microphone may offer advantages over traditional ultrasound transducers used to measure ultrasound waveforms. The MEMS microphone may provide a wider field of view (FOV) for receiving acoustic signals and may have a smaller form factor compared to conventional transducers, making it beneficial for use in a wearable device. For example, in some variations, the one or more MEMS microphones may be configured to receive an acoustic waveform with a field of view between about 60 degrees to about 270 degrees, about 90 degrees to about 180 degrees, including all subranges and values therebetween. A sensor comprising a plurality of MEMS microphones may include a processor configured to select a MEMS microphone or subset the plurality of microphones to measure the reflected acoustic waveform. The selection may be based on a signal to noise ratio (SNR) of the reflected acoustic waveform measured by the MEMS microphone compared to other MEMS microphones of the plurality of MEMS microphones. Alternatively, the measurements of all MEMS microphones of the plurality of microphones may be processed together (e.g., combined) to generate voice data.

### Support

Generally, the support (150) of a vocal data generation system (100) may be configured to couple (e.g., attach, affix) to one or more transducers (104) in a compact and lightweight form factor that may be comfortably and releasably coupled to a subject while the vocal data of the subject is being generated. The support may allow the sensor (102) to remain substantially fixed relative to the subject even when the subject moves one or more portions of their body (e.g., head turns, walks, stands up, lies down). In some variations, the support (150) may have a shape configured to conform to one or more of a shape (e.g., curvature) of the shoulders, chest, and back of a subject. For example, the support (150) may comprise a concave shape and/or may be compliant to form a concave shape when placed in contact with subject anatomy (e.g., on the shoulders). The shape, contour, and size of the support may be selected based on a size of the subject (e.g., adult, child), and in some variations, the shape, contour, and/or size of the support may be configured for a demographic group (e.g., adults, children, male, female). In some variations, the support may comprise a concave shape configured to releasably couple to one or more of a neck, a shoulder, a back, and a chest of a subject.

Additionally or alternatively, the support (150) may comprise one or more of a collar, a cuff, a strap, a fastener, a clamp, a line, and an anchor configured to releasably secure the sensor (102) to the subject. For example, a collar may be coupled to the transducer (104) and rest on the shoulder(s) of the subject. One or more end portions of the support (150) may comprise a clamp configured to apply inward pressure against the support (150) and subject to secure (e.g., hold) the support (150) to the subject.

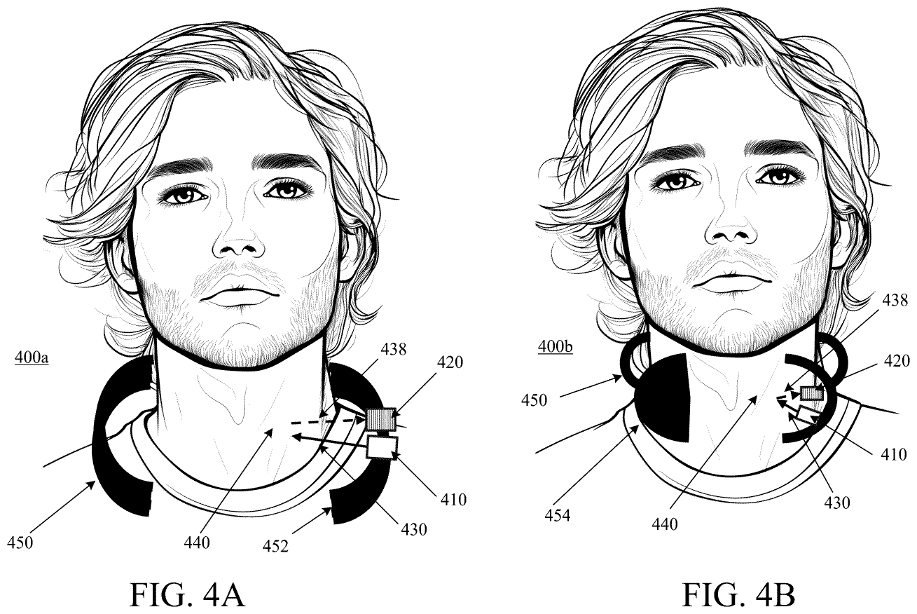

In some variations, a support may be configured to position a transducer relative to a vibrating surface to measure the voice signal of the subject. FIG. 4A is a schematic side view of an illustrative variations of a sensor (400a) comprising one or more transducers (410, 420) coupled to a support (450) configured to releasably coupled the sensor (400a) to the subject. For example, as shown in FIG. 4A, the support (450) (e.g., collar) may be configured to releasably couple to one or more of the shoulders, chest, or back of the subject. In some variations, the support (450) may define a thickness (452) configured to position the one or more transducers (410, 420) to transmit and measure an acoustic waveform (430, 438) reflected by a vibrating surface of the subject (440). The thickness (452) of the support (450) may be selected based on the particular anatomy of the subject and/or the location on the vibrating surface to be measured (e.g., skin of the neck covering the larynx).

In some variations, the support (450) may house one or more transducers (410, 420). FIG. 4B is a schematic side view of an illustrative variations of a sensor (400b) comprising one or more transducers (410, 420) coupled to a support (450) configured to releasably coupled the sensor (400a) to the subject. As show in FIG. 4B, the support (450) (e.g., collar) may be configured to releasably couple to the neck of the subject in order to position one or more transducers (410, 420) to transmit an acoustic waveform (430) and measure a reflected acoustic waveform (438). In some variations, the support (450) may comprise a housing (454) configured to house one or more transducers (410, 420) and one or more other elements of the sensor (400b). In some variations, the housing (454) may be configured to shield the one or more transducers (410, 420) from undesirable noise.

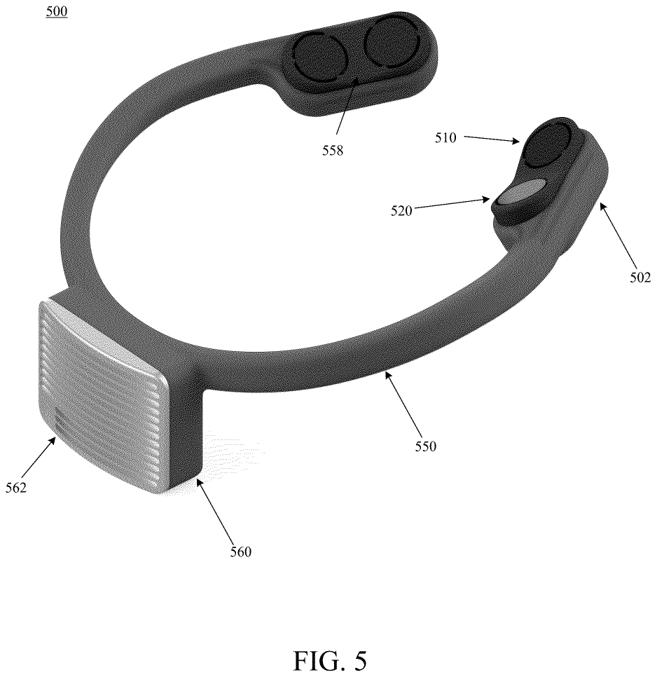

Generally, the support of a vocal data generation system (100) may be configured to facilitate the generation of vocal data in a compact and lightweight form factor that may be comfortably and releasably coupled to a subject. FIG. 5 is a perspective view of an illustrative variation of a sensor (500) comprising a support (550) coupled to one or more transducers (502). In some variations, the support (550) may comprise one or more of a C-shape, a U-shape, a horseshoe-shape, and a concave shape configured to releasably couple to one or more of a neck, a shoulder, a back, and a chest of a subject. As shown in FIG. 5, the support may comprise one or more of a transducer housing (558) and a component housing (560). The transducer housing (558) may be configured to position one or more transducers (502) to transmit acoustic waveforms or measure acoustic waveforms from the subject. In some variations, the transducer housing (558) may be configured to adjust an angle of a transducer configured to transmit acoustic waveforms (510) or a transducer configured to measure reflected acoustic waveforms (520) based on the anatomy of the subject.

In some variations, the support (550) may comprise a component housing (560) configured to house various other components of a vocal data generation system in a compact form factor. Other components of the vocal data generation system may include one or more of a power source, a processor, a memory, an input device, an output device, a signal generator, and a communication device. In some variations, the component housing (560) may comprise a partially open face (562) configured to allow heat transfer between the components within and the environment and/or facilitate an interaction between the subject and the input/output device.

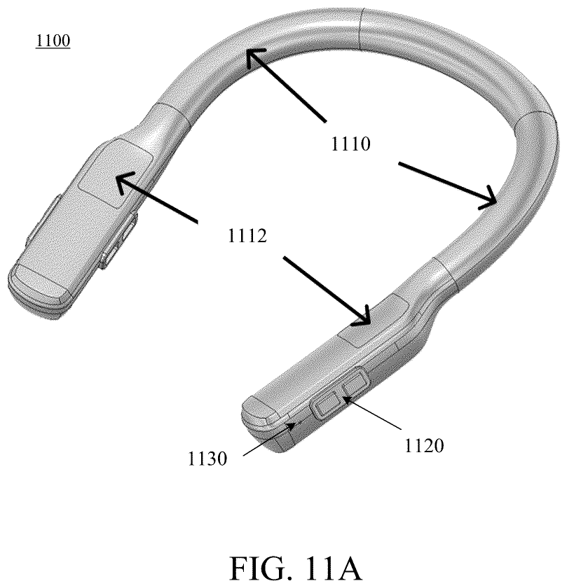

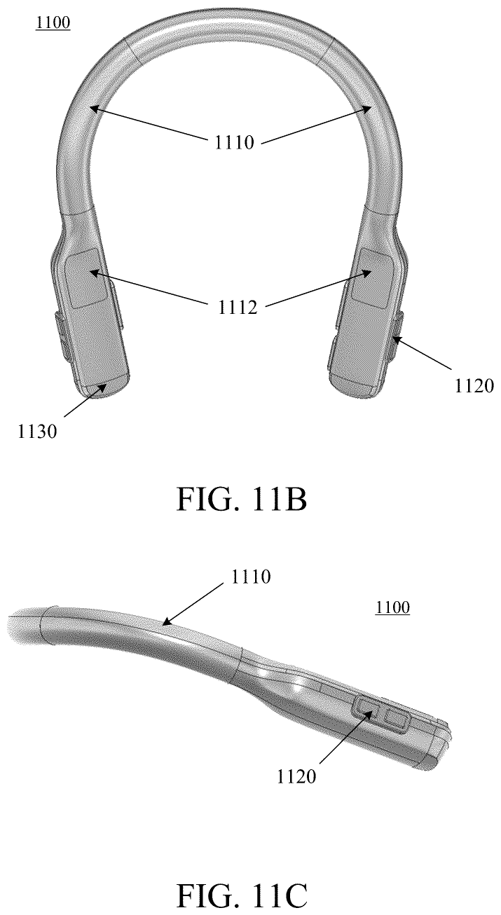

FIGS. 11A, 11B, and 11C are a perspective view, a top view, and a side view respectively of another illustrative variation of a wearable sensor (1100) comprising a support (1110) coupled to one or more transducers (1112). In some variations, the support (1100) may comprise one or more of a C-shape, a U-shape, a horseshoe-shape, and a concave shape configured to releasably couple to one or more of a neck, a shoulder, a back, and a chest of a subject. The support (1110) may be configured to position one or more transducers (1112) configured as transmitters to transmit acoustic waveforms or measure acoustic waveforms from the subject. Additionally or alternatively, the support (1110) may position one or more transducers configured as receivers (1130) to measure a reflected acoustic waveform. In some variations, as shown in FIGS. 11A, 11B, and 11C, one or more transducers configured as receivers may be MEMS microphones. The transducer configured as a receiver (1130) may be positioned on a surface of the support (1110) facing the subject as shown in FIG. 11B. In other variations, where the transducer has a wide field of view, for example if the receiver is a MEMS microphone, the transducer configured as a receiver may be positioned on a surface of the support (1110) facing away from the subject. The support (1110) may comprise one or more input/output devices (1120) for controlling the sensor (1100). For example, as shown in FIGS. 11A-11C, the sensor (1100) may comprise one or inputs (e.g., buttons, switches) for, for instance, increasing the intensity of an audio output device, powering the sensor (1100) on or off, and/or starting and/or stopping measuring and generation of vocal data from the subject.

### Signal Generator

Generally, the signal generators described herein may be coupled to one or more transducers (e.g., transducer array) and configured to provide energy (e.g., signal waveforms, acoustic waveform) to one or more of the transducers to transmit to the subject (e.g., skin of the neck). In some variations, a vocal data generation system (100) as described herein may include a signal generator (170) operatively coupled to a power source (162) and a processor (164). In some variations, the signal generator may comprise a multiplexer configured to select one or more transducers of a transducer array to transmit a signal waveform (e.g., acoustic waveform) generated by the signal generator according to a predetermined sequence. The multiplexer of the signal generator may be configured to independently address the transducers of the transducer array.

In some variations, a signal generator may be operatively coupled to one or more transducers. The signal generator may be configured to generate the signal waveform. In some variations, the signal waveform (e.g., acoustic waveform) may comprise a frequency of between about 40kHz to about 300 kHz, between about 40 kHz to about 200 kHz, between about 40 kHz to about 100 kHz, between about 100 kHz to about 300 kHz, between about 100 kHz to about 200 kHz, and between about 150 kHz to about 250 kHz, including all subranges and values therebetween. For example, the signal waveform may comprise a frequency of about 40 kHz, about 80 kHz, about 100 kHz, about 150 kHz, about 200 kHz, about 250 kHz or about 300 kHz. In some variations, the signal generator may be configured to control signal waveform generation and transmission in response to measured reflected signal waveforms. For example, a field of view of the acoustic waveform may be modified based on measured reflected signal waveform data, as will be described in more detail herein.

### Input Device and Output Device

Generally, an input device of a vocal data generation system may serve as a control interface for a subject (e.g., user, wearer). In some variations, the system may comprise one or more input devices. For example, the system (100) may comprise an input/output device (168) (e.g., button switch) configured to control the system (100). Additionally or alternatively, the computing device (180) may comprise a corresponding input device (e.g., touchscreen interface) configured to control the system (100). In some variations, the input/output device (168) may be configured to receive input to control one or more of the signal generator (170), the communication device (172), and the like. For example, subject actuation of an input/output device (168) (e.g., switch) may be processed by the processor (164) and the memory (166) to output a control signal to signal generator (170).

Some variations of an input device may comprise at least one switch configured to generate a control signal. In some variations, the switch may comprise a single button. In some variations, the switch may comprise a plurality of buttons, for example, located at different portions of the sensor (102).

Additionally or alternatively, in variations of an input/output device (168) comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive a signal from an optical sensor and classify a patient gesture as a control signal. A microphone may be configured to receive audio and recognize a voice (e.g., verbal command) as a control signal. In variations of a system comprising a plurality of input devices, different input devices may generate different types of signals. For example, some input devices (e.g., button on sensor) may be configured to generate a control signal to start/stop vocal data generation while other input devices (e.g., touchscreen of computing device) may be configured to generate a control signal to modify signal waveform parameters.

Generally, an output device of a vocal data generation system (100) and/or computing device (180) may be configured to output data corresponding to the system (100) (e.g., vocal data, response to vocal data), and may comprise one or more of a display device (e.g., set of LEDs), audio device (e.g., speaker), and haptic device. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

In some variations, the input/output device (168) may comprise one or more LEDs (e.g., one, two, three, four, or more) and may include a tricolor LED (e.g. red, green, blue). In some variations, the input/output device (168) may be configured to indicate, for example, a status of the sensor (102). For example, the output device 130 may be configured to indicate one or more of an operation state (e.g., recording vocal data), a standby state, a battery charge state (e.g., low, charged, charging, voltage value), an alert state, an error state, and a computing device connection state.

In some variations, the input/output device (168) may comprise an optical waveguide (e.g., light pipe, light distribution guide, etc.). One or more optical waveguides may receive light from a light source (e.g., illumination source) using a predetermined combination of light output parameters (e.g., wavelength, frequency, intensity, pattern, duration). In some variations, the optical waveguide may be formed integrally with one or more portions of a housing of the sensor (102). An optical waveguide may refer to a physical structure that guides electromagnetic waves such as visible light spectrum waves to passively propagate and distribute received electromagnetic waves. Non-limiting examples of optical waveguides include optical fiber, rectangular waveguides, light tubes, light pipes, combinations thereof, or the like. For example, light pipes may comprise hollow structures with a reflective lining or transparent solids configured to propagate light through total internal reflection. The optical waveguides described herein may be made of any suitable material or combination of materials. For example, in some variations, the optical waveguide may be made from optical-grade polycarbonate. In some variations, the optical waveguides described herein may comprise one or more portions configured to emit light. In some variations, the optical waveguides described herein may comprise a surface contour including, for example, a multi-faceted surface configured to increase visibility from predetermined vantage points.

The light patterns described herein may, for example, comprise one or more of flashing light, occulting light, isophase light, etc., and/or light of any suitable light/dark pattern. For example, flashing light may correspond to rhythmic light in which a total duration of the light in each period is shorter than the total duration of darkness and in which the flashes of light are of equal duration. Occulting light may correspond to rhythmic light in which the duration of light in each period is longer than the total duration of darkness. Isophase light may correspond to light which has dark and light periods of equal length. Light pulse patterns may include one or more colors (e.g., different color output per pulse), light intensities, and frequencies.

In some variations, the vocal data generation system may additionally or alternatively comprise an output device such as an audio output device and/or a haptic device. For example, an audio output device may audibly output vocal data, reflected waveform data, error data, system data (e.g., power source status), alarms, notifications, and/or responses. For example, the audio output device may output an audible alarm when a power source has insufficient power. In some variations, an audio device may comprise at least one of a speaker, a piezoelectric audio device, a magnetostrictive speaker, and/or digital speaker. In some variations, a user may communicate with other subjects using the sensor or traditional voice recording device (e.g., microphone) and a communication channel. For example, a user may form an audio communication channel (e.g., cellular call, VoIP call) with another person. In some variations, a user may communicate using the sensor with a natural language model, machine learning model, or other computer algorithm.

In some variations, an audio output device of input/output device (168) and/or computing device (180) may be configured to indicate a status of the sensor (102). For example, the audio output device (and any of the output devices described herein) may be configured to indicate an operation state (e.g., generating vocal data), a sleep state, a standby state, a battery charge state (e.g., low, charged, charging, voltage value), an alert state, an error state, and a computing device connection state.

In some variations, a haptic device may be incorporated into the vocal data generation system (100) and/or computing device (180) to provide additional sensory output (e.g., force feedback) to the user. For example, a haptic device may generate a tactile response (e.g., vibration) to confirm user input to an input device (e.g., button) or to communicate an operation state (e.g., first vibration pattern corresponding to a first operation state, second vibration pattern corresponding to a second operation state).

### Processor and Memory

A vocal data generation system (100), as depicted in FIG. 1, may comprise a processor (164) and a machine-readable memory (166) (e.g., collectively a controller) in communication with one or more computing devices (180). The processor (164) may be connected to the computing devices (180) by wired or wireless communication channels. The processor (164) may be configured to control one or more components of the system (100), such as the signal generator (170), the transducer (102), the input/output device (168), and the communication device (172). The processor (164) may be implemented consistent with numerous general purpose or special purpose computing systems or configurations. Various exemplary computing systems, environments, and/or configurations that may be suitable for use with the systems and devices disclosed herein may include, but are not limited to software or other components within or embodied on personal computing devices, network appliances, servers or server computing devices such as routing/connectivity components, portable (e.g., hand-held) or laptop devices, multiprocessor systems, microprocessor-based systems, and distributed computing networks.

The processor (164) may incorporate data received from the memory (166), input/output device (168), the transducer (102) and computing device (180) to control the system (100). The memory (166) may further store instructions to cause the processor (164) to execute modules, processes, and/or functions associated with the system (100) and/or computing device (180). The processor (164) may be any suitable processing device configured to run and/or execute a set of instructions or code and may comprise one or more microcontrollers, data processors, image processors, graphics processing units, physics processing units, digital signal processors, and/or central processing units. The processor (164) may be, for example, a general purpose processor, a Field Programmable Gate Array (FPGA), an Application Specific Integrated Circuit (ASIC), configured to execute application processes and/or other modules, processes, and/or functions associated with the system and/or a network associated therewith. For example, the processor (164) may be a dual core microcontroller. The underlying device technologies may be provided in a variety of component types such as metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, combinations thereof, and the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the processor (164) may be configured to process a measured reflected acoustic waveform to generate vocal data. The vocal data may include audio vocal data and voice to text data. In some variations, the processor (164) may be configured to generate vocal data based on estimated in change in the measured reflected acoustic waveform compared to the transmitted acoustic waveform parameters. For example, the processor (164) may be configured to generate vocal data based on an estimated change in one or more of phase and frequency of the measured reflected acoustic waveform.

In some variations, the processor (164) may be configured to perform additional operations on generated vocal data. In some variations, the processor may be configured to generate a response to vocal data by applying one or more of a machine learning model and a natural language model to the vocal data. In some variations, the processor may be configured to output to the subject a response using the output device or communication device based on the application of a of a machine learning model and/or a natural language model.

In some variations, the memory (166) may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions associated with the communication device, such as waveform data processing, vocal data generation, phase or frequency change estimation, sensor control, machine learning algorithm application, natural language model application, and/or communication. Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CD-ROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

### Communication Device and Network

In some variations, vocal data generation systems (100) described herein may communicate with networks (182) and computer systems through a communication device (172). In some variations, the vocal data generation system (100) may be in communication with other devices (e.g., computing devices) via one or more wired and/or wireless networks (182). A wireless network may refer to any type of digital network that is not connected by cables of any kind. Examples of wireless communication in a wireless network include, but are not limited to Bluetooth, cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

The communication device (172) may comprise RF circuitry configured to receive and send RF signals. The RF circuitry may convert electrical signals to/from electromagnetic signals and communicate with communications networks and other communications devices via the electromagnetic signals. The RF circuitry may comprise well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

Wireless communication through any of the computing and measurement devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

In some variations, communication using the communication device (172) may be encrypted. Any of the data stored in memory (166) (e.g., respiratory parameter data described herein) may be transmitted using the communication device (172)).

### Power Source

Generally, the vocal data generation systems described herein may receive power from an internal power source (e.g., lithium battery, disposable battery) and may optionally be recharged using an external power source (e.g., wireless charger, wall outlet, computing device). In some variations, the vocal data generation system may additionally or alternatively receive power via a wired connection, and/or a wireless connection (e.g., induction, RF coupling, etc.). The vocal data generation system may comprise one or more power algorithms configured to conserve energy and increase a lifespan of the vocal data generation system. Additionally or alternatively, the sensor (102) may be coupled to an external power source.

### Computing Device

Generally, the computing device (180) described here may comprise a controller comprising a processor (e.g., CPU) and memory (which can include one or more non-transitory computer-readable storage mediums). The processor may incorporate data received from memory and over a communication channel to control one or more components of the system (e.g., sensor (102)). The memory may further store instructions to cause the processor to execute modules, processes and/or functions associated with the methods described herein. As used herein, a computing device may refer to any of the computing devices (180) and databases (190) as depicted in FIG. 1. In some variations, the memory and processor may be implemented on a single chip. In other variations, they can be implemented on separate chips.

A controller may be configured to receive and process one or more of vocal data and reflected signal waveform data from the sensor (102) and other data from other sources (e.g., computing device (180), database (190), user input). The computing device may be configured to receive, process, compile, store, and access data. In some variations, the computing device may be configured to access and/or receive data from different sources. The computing device may be configured to receive data directly input and/or measured from a user. Additionally or alternatively, the computing device may be configured to receive data from separate devices (e.g., a smartphone, tablet, computer) and/or from a storage medium (e.g., flash drive, memory card). The computing device may receive the data through a network connection, as discussed in more detail herein, or through a physical connection with the device or storage medium (e.g. through Universal Serial Bus (USB) or any other type of port). The computing device may include any of a variety of devices, such as a cellular telephone (e.g., smartphone), tablet computer, laptop computer, desktop computer, portable media player, wearable digital device (e.g., digital glasses, wristband, wristwatch, brooch, armbands, virtual reality/augmented reality headset), television, set top box (e.g., cable box, video player, video streaming device), gaming system, or the like.

The computing device may be configured to receive various types of data. For example, the computing device may be configured to receive a natural language model or other machine learning model including numerical parameters and framework data. While the above mentioned information may be received by the computing device, in some variations, the computing device may be configured to process any of the above data from information it has received using software stored on the device itself, or externally.

### II. Methods

Also described here are methods for generating vocal data from a subject with reduced noise using the systems and devices described herein. In particular, the systems, devices, and methods described herein may be used to accurately record the voice of a subject in real-time, such as, for example, vocal data. The methods described herein are advantageous relative to conventional methods in several ways. Traditional ultrasound signal processing relies on a time of flight of the signal as opposed to changes in phase or frequency of the signal. For example, traditional ultrasound imaging relies on pulsed waves of ultrasound. However, in order to measure vocal data, a more continuous signal is desirable, or else the vocal data may be incomplete. That is, the portions of the continuous vocal signal produced during the gaps between pulses of a traditional ultrasound signal may not be measured. Other traditional methods of vibration monitoring such as laser vibrometry require precise calibration and are sensitive to displacement of the sensor, imperfect reflection from skin, and inadequate lighting conditions. If these precise conditions are not met, the vibration signal cannot be accurately generated. Thus, these methods are unsuitable incorporation into a wearable device. By contrast, the methods of the present invention are more robust and less sensitive.

For example, the sensors described herein, continuously or semi-continuously, and non-intrusively monitor vocal data of a subject from the shoulder of the subject. Moreover, because the sensor is compact and may measure the vibrations of a surface on a subject without physically contacting the surface, the sensor may remain comfortably and continuously wearable for several hours or days without interfering with the subject. Additionally, while calibration of the sensor may be used if so desired, the methods described herein do not rely on or require calibration for placement of the sensors or focusing of signal waveforms, and thus the number of steps, complexity, and time required to utilize the vocal data generation devices, systems, and methods described herein may be reduced.

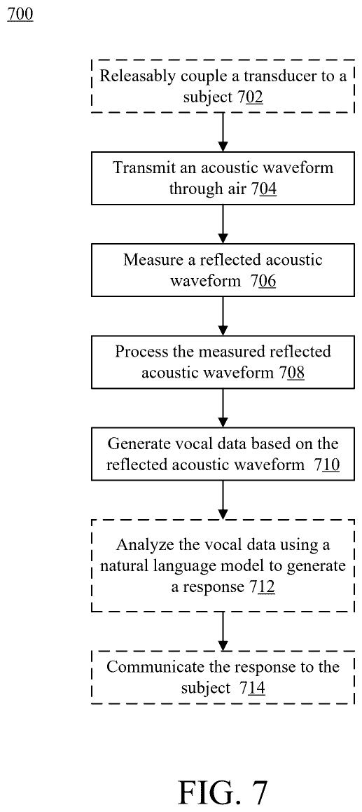

Generally, the methods using any of the devices and systems described herein may include the steps of transmitting an acoustic waveform using a transducer to a vibrating surface of a subject where the subsequent the reflected acoustic waveforms may be measured by the same or different transducer. Vocal data may be generated based on the measured reflected acoustic waveforms. For example, FIG. 7 depicts a flowchart representation that generally describers a method of generating vocal data from a subject (700). In some variations, the method (700) may include releasably coupling a transducer of a sensor to a subject (702). For example, a support (150) may be releasably coupled to the subject at the shoulder, the back, and/or the chest of the subject. Releasably coupling the sensor to one or more of these locations may improve adoption of the sensor and increase the time the sensor can be worn comfortably. Additionally or alternatively, the support (150) may be configured to releasably couple to the neck of the subject such that the transducer (104) is not in physical contact with the neck. Releasably coupling the sensor to this location may improve the compactness of the sensor.

The method (700) may include transmitting an acoustic waveform through air to a skin of a subject (704). In some variations, the transmitting the acoustic waveform may comprise transmitting the acoustic waveform through air or other gaseous medium to take advantage of a difference in impedance between the subject and the air or other gaseous medium. The acoustic waveform may be transmitted to the skin of a subject including one or more of the skin of the neck, the jaw, and chin. In particular, the skin of the neck may comprise the skin covering one or more of the larynx, the circloid cartilage, the thyroid cartilage, the thyroid gland, the trachea, and the lymph nodes. In some variations, the acoustic waveform is transmitted from between about 5 cm and about 15 cm away from the skin of the subject, between about 5 cm and about 10 cm away from the skin of the subject, and between about 2 cm and about 7 cm away from the skin of the subject, including all subranges and values therebetween. For example, transducer (510) in FIG. 5 may be disposed between about 2 cm and about 7 cm away from a throat of a subject.

In some variations, the transmitted acoustic waveform may comprise one or more predetermined parameters to facilitate coupling with the voice signal of the subject in the reflected waveform. In some variations, the acoustic waveform may be transmitted at a predetermined frequency to provide sufficient bandwidth to capture a voice signal and avoid capturing noise in a reflected acoustic waveform. For example, transmitting the acoustic waveform at a higher frequency may improve the quality of the generated vocal data and enable more variations for the processing the vocal data. In some variations, the acoustic waveform (e.g., ultrasound pressure wave) may be transmitted at a frequency of between of about 40kHz to about 300 kHz, about 40 kHz to about 200 kHz, about 40 kHz to about 100 kHz, about 100 kHz to about 300 kHz, about 100 kHz to about 200 kHz, and about 150 kHz to about 250 kHz, including all subranges and values therebetween. For example, the acoustic waveform may be transmitted to the skin of the subject at a single frequency of about 40 kHz, about 80 kHz, about 100 kHz, about 150 kHz, about 200 kHz, about 250 kHz or about 300 kHz. In some variations, the acoustic waveform may be transmitted at one or more different frequencies to improve signal quality and robustness of reflect signal. For instance, one or more transducers may each transmit an acoustic signal at a different frequency. Each frequency may differ from other frequencies by at least about 5 Hz, at least about 10 Hz, at least about 50 Hz, at least about 100 Hz, at least about 500 Hz, or at least about 1000 Hz, including all subranges and value therebetween. Each frequency may be received by a single broadband receiver (e.g., MEMS microphone). Alternatively, each frequency may be received by a different transducer configured to measure the respective frequency.

In some variations, the acoustic waveform may be transmitted with a predetermined strength (e.g., amplitude) and field of view to reflect off the vibrating surface of the subject. For example, the acoustic waveform may be transmitted at a sound pressure level of between about 90 dB and about 135 dB at about 30 cm, between about 90 dB and about 120 dB at about 30 cm, between about 105 dB and about 135 dB at about 30 cm, and between about 105 dB and about 120 dB at about 30 cm. For example, the acoustic waveform may be transmitted at a sound pressure level of about 90 dB, about 100 dB, about 110 dB, about 115 dB, about 125 dB, and about 135 dB at about 30 cm, including all subranges and values therebetween. In some variations, the acoustic waveform may be transmitted to comprise a field of view broad enough to target one or more vibrating surfaces of the subject. The acoustic waveform transmitted with a broad field of view may reflect off one or more vibrating surfaces and return to the transducer or a separate transducer configured as a receiver. A broad field of view may reduce the amount of time or eliminate steps needed to calibrate the sensor. For example, the acoustic waveform may be transmitted with a field of view of between about 30 degrees and about 120 degrees. For example, the acoustic waveform may be transmitted with a field of view of about 30 degrees, about 50 degrees, about 70 degrees, about 90 degrees, and about 120 degrees. In some variations, the method (700) may further comprise a calibration step involving selecting one or more of the above acoustic waveform parameters based on one or more of a distance between the transducer and the skin of the subject, the anatomy of the subject, known qualities of the voice signal of the subject, and known acoustic qualities of the environment.

The method (700) may include measuring an acoustic waveform (706) reflected by the skin of the subject. In some variations, the reflected acoustic waveform may be reflected by the skin of the subject and comprise the voice signal of the subject. For example, the reflected acoustic waveform may be modulated to contain the voice signal by vibrations in the skin caused by speech by the subject at the time of reflection. The transmitted and reflected acoustic waveforms may propagate through space without substantial modulation from the environment. The reflected acoustic waveform may be reflected by the skin of a subject including one or more of the skin of the neck, the jaw, and chin. In some variations, the reflected acoustic waveform may be measured from between about 5 cm and about 15 cm away from the skin of the subject, between about 5 cm and about 10 cm away from the skin of the subject, and between about 2 cm and about 7 cm away from the skin of the subject, including all subranges and values therebetween. In some variations, the acoustic waveform may be transmitted from a first location and the reflected acoustic waveform may be measured at a second, different location. In some variations, the reflected acoustic waveform may comprise a scattered acoustic waveform caused by the acoustic waveform diffusing against the skin of the subject.

In some variations, in which a broadband transducer (e.g., MEMS microphone) is used as a receiver, environmental sounds may be measured by the transducer simultaneously with the reflected acoustic waveform. This may cause the measured reflected acoustic signal to comprise additional modulations from environmental sounds. These modulations may be introduced at the receiver and are introduced by vibrations in a membrane of the receiver caused by the environmental sounds. This may undermine the ability to isolate vibrations in the subject in the modulated signal. Thus, in some variation, the measured signal may be processed to remove lower frequency environmental modulations. For example, the measured reflected acoustic waveform may be measured digitally or converted to a digital signal and filtered to reduce environmental modulations. In some variations, a mechanical filter may be used to reduce environmental (e.g., baseband) interference.

However, in some variations, where the environmental sounds comprise vocal data from the subject, such as the voice of the subject, modulations from those environmental sounds may be advantageous. For example, a broadband receiver (e.g., MEMS microphone) may measure a reflected acoustic waveform modulated by both the vibrations of the reflecting surface of the subject and the voice of the subject. The reflected acoustic waveform may be processed (e.g., demodulated) as described herein to extract a first voice signal corresponding to the modulations caused by the reflecting surface and a second voice signal corresponding to the modulations caused by the voice of the subject. The first and second voice signals may be combined to generate a combined voice signal with improved signal quality and improved signal to noise ratio. In some variations, the first and second signals may be combined to remove noise introduced from environmental sounds without removing the voice signal.

As described above, in some variations, transmitting the acoustic waveform and measuring the reflected acoustic waveform may use at least one transducer. The transducer may be configured to transmit acoustic waveforms to measure reflected acoustic waveforms, as described above. In some variations, measuring the reflected acoustic waveform may use a transducer comprising a sensitivity of between about 0.01 V/Pa and about 100 V/Pa, including all subranges and values therebetween. In some variations, calibrating the systems, devices, and methods described herein may involve selecting or adjusting a transducer based on one or more of a distance between the transducer and the skin of the subject, the anatomy of the subject, predetermined qualities of the voice signal of the subject, and predetermined acoustic characteristics of the environment.

In some variations, one or more of transmitting the acoustic waveform (704) and measuring the reflected acoustic waveform (706) may be performed continuously to generate continuous vocal data. For example, continuously transmitting the acoustic waveform may include transmitting an uninterrupted waveform (e.g., sine wave) at a predetermined frequency for a predetermined time period. In some variations, continuously transmitting the acoustic waveform and continuously measuring the reflected acoustic waveform may be performed over a predetermined time period beginning upon a first input from the subject (e.g., button press, activation signal) and ended by a second input from the subject (e.g., button press, deactivation signal). By contrast, conventional solutions transmit pulsed (e.g., discontinuous) waveforms incapable of providing continuous vocal data, thereby rendering conventional solutions ineffective for generating vocal data.

The method (700) may include processing the measured reflected acoustic waveform (708) to generate vocal data based on the reflected acoustic waveform (710). Generally, processing the measured reflected acoustic waveform (708) to generate vocal data (710) may involve extracting a voice signal from the measured reflected acoustic waveform. In some variations, processing the measured reflected acoustic waveform may comprise estimating a change in one or more of a phase and a frequency of the measured reflected acoustic waveform. In some variations, processing the measured reflected waveform comprises processing a scattered acoustic waveform. Processing the measured reflected acoustic waveform may include applying one or more of quadrature demodulation, a phase lock loop, a Hilbert transformation, offset demodulation, and/or zero-cross demodulation to demodulate the reflected acoustic waveform and extract vocal data.

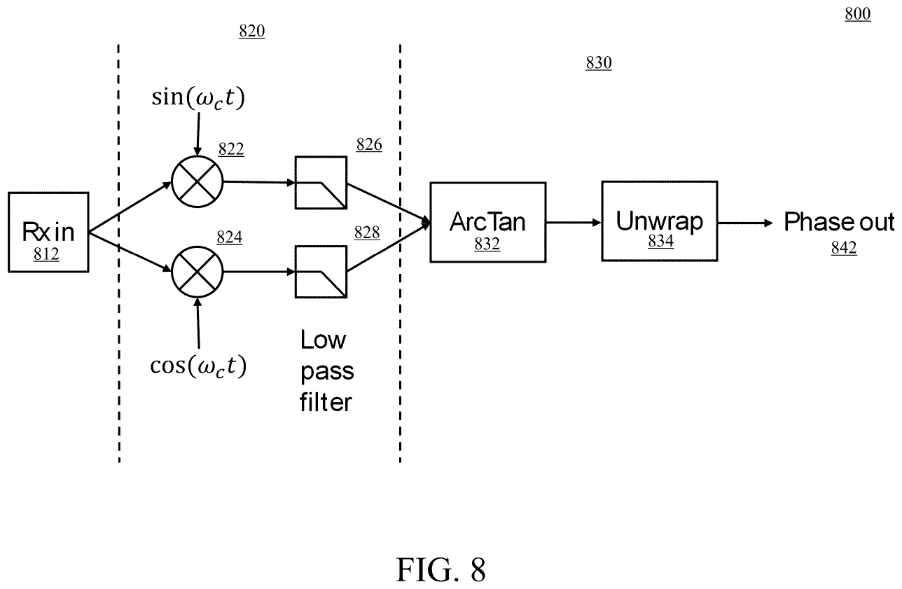

Processing the measured reflected acoustic waveform to generate vocal data may include applying a processing method (e.g., pipeline) to generate vocal data. In some variations, the processing method may receive an input signal (e.g., the measured reflected acoustic waveform) filtered to remove baseband signals. The input signal may be one or more of an analog or digital signal. In some variations, processing the measured reflected acoustic waveform may comprise applying a quadrature demodulation to the measured reflected acoustic waveform. For example, FIG. 8 is a flowchart describing an illustrative variation of a method of processing a signal waveform using quadrature demodulation. The processing method (800) may comprise receiving an input signal (812), applying a quadrature demodulation (820), and further processing of the modulated signal (830) (e.g., voice signal) to generate phase out (942) (e.g. voice data). Quadrature demodulation may involve splitting the input signal into a quadrature signal (822) (e.g., sine component) and in-phase signal (824) (e.g., cosine component) and applying a low pass filter (826, 828) to each signal (822, 824). The quadrature signal (822) and in-phase signal (824) may be recombined by applying an arctangent function (832). An unwrap function (834) may then be applied to remove undesired phase shifts and output (e.g., estimate, recover) an original phase (842). In some variations, the signal may be converted from analog to digital before applying a quadrature demodulation (820).

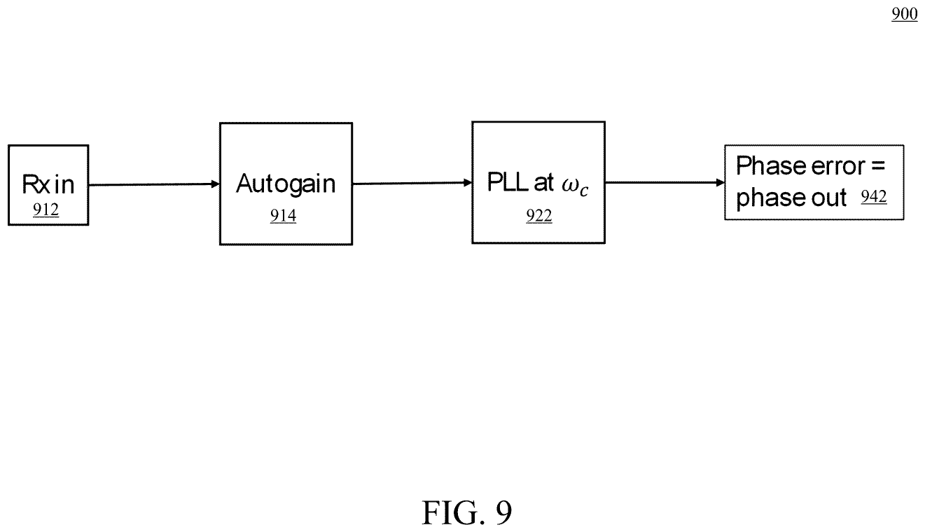

In some variations, processing the measured reflected acoustic waveform comprises inputting the measured reflected acoustic waveform to a phase lock loop circuit to estimate a phase of the measured reflected acoustic waveform. For example, FIG. 9 is a flowchart describing an illustrative variation of a method of processing a signal waveform using a phase lock loop. The processing method (900) may comprise receiving an input signal (912), applying an autogain function (914), and inputting the signal to a phase lock loop circuit (922) configured to the carrier frequency to generate an output phase (942) (e.g. phase error, voice data). Applying the autogain function (914) may stabilize (e.g., normalize) the input signal to allow the phase-locked loop circuit (922) to extract the phase. In some variations, the phase error (i.e. the feedback signal of the phase-locked loop circuit) may be the desired output of the processing method (900) and correspond to the vocal data. In some variations, the input signal (912) may be converted to a digital signal and the phase lock loop circuit (922) may be applied in software.

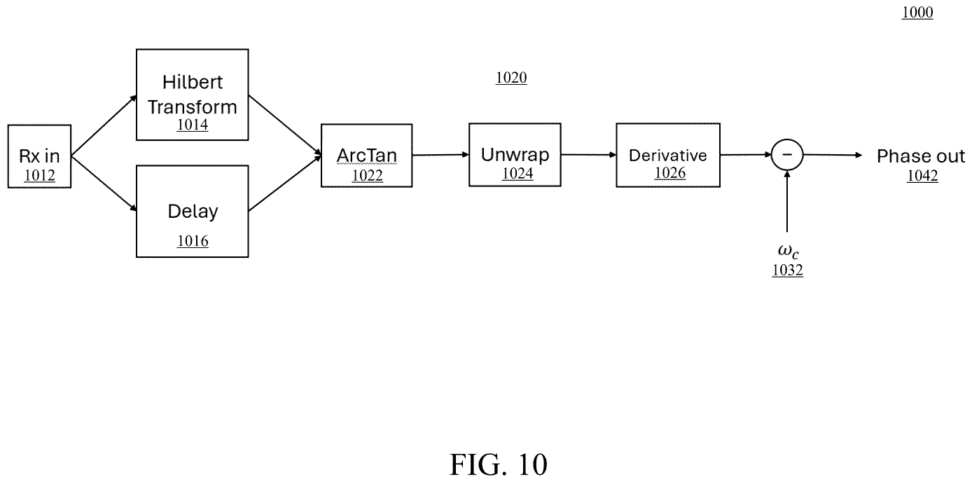

In some variations, processing the measured reflected acoustic waveform may comprise applying a Hilbert transformation to estimate a phase of the measured reflected acoustic waveform. For example, FIG. 10 is a flowchart describing an illustrative variation of a method of processing a signal waveform applying a Hilbert transformation. The processing method (1000) may comprise receiving an input signal (1012), applying a Hilbert transformation (1014) to the input signal and reserving a delay signal (1016), further processing (1020) of the modulated signal (e.g., voice signal), and subtracting out a carrier frequency signal (1032) to generate an output phase (1042) (e.g. voice data). In some variations, the processing method (1000) may receive the input signal (1012) as a digital signal and process the input signal (1012) using software. Applying the Hilbert transformation (1014) along with the delay (1016) signal may result in a quadrature signal and an in-phase signal. The Hilbert transformation (1014) signal and delay (1016) signal may be recombined by applying an arctangent function (1022) to create a signal comprising vibrations carried by the reflected acoustic waveform. An unwrap function (1034) may be applied to remove undesired phase shifts. A derivative function (1026) may be applied to the signal to remove unwanted drift. The carrier frequency signal (1032) (e.g., acoustic waveform signal) may be subtracted to generate a phase corresponding to the vocal data (1042). Additionally or alternatively to subtracting the carrier frequency signal (1032), a high pass filter (not shown) may be applied to the signal to generate the phase corresponding to the vocal data.

In some variations, processing the measured reflected waveform may include offset demodulation to extract the vocal data. Traditional frequency modulation (FM) demodulation assumes a relatively large modulation depth. However, a modulation depth of the measure reflected acoustic signal may be relatively small. For example, the modulation depth of the measured reflected acoustic waveform may be about 1/10⁴ to about 1/10⁵. Unexpectedly and surprisingly, offset demodulation may increase the resolution of the processed reflected acoustic signal and improve the signal to noise ratio of the resulting signal. For example, applying an offset demodulation with an offset of about 500 Hz to about 10 kHz or about 1 kHz to about 4 kHz, including all subranges and values therein, from carrier frequency may improve the quality of the extracted vocal data.

In some variations, the output of a processing method may be processed further to improve the quality of the vocal data or generate a different form of vocal data. For example, the method (700) may further comprise applying an equalizer to the vocal data (e.g., audio voice data, output phase) to modulate the vocal data to better resemble the direct vocal sounds produced by the subject. Additionally or alternatively, the method may include transcribing the vocal data to text data, and communicating the vocal data using a communication device to one or more of the subject, another subject, and a computing device.

In some variations, the method (700) may include applying a natural language model (712) to the generated vocal data to generate a response (e.g., output audio, output text). The natural language model may be or comprise one or more of autoregressive language models, neural language models, Bidirectional Encoder Representations from Transformers, N-gram models, rule-based models, machine translation models, sentiment analysis models, text summarization models, and parsing model. In some variations, the method (700) may include inputting the vocal data to a machine learning model and generating a response using the machine learning model. Additionally or alternatively, the vocal data may be input into the machine learning model to train the model. In some variations, the method (700) may include communicating the response to the subject (714). For example, the response may comprise audio data communicated to the subject using any one of the output devices described above including a speaker.

Although the foregoing variations have, for the purposes of clarity and understanding, been described in some detail by illustration and example, it will be apparent that certain changes and modifications may be practiced and are intended to fall within the scope of the appended claims. Additionally, it should be understood that the components and characteristics of the systems and devices described herein may be used in any combination. The description of certain elements or characteristics with respect to a specific figure are not intended to be limiting or nor should they be interpreted to suggest that the element cannot be used in combination with any of the other described elements. For all of the variations described herein, the steps of the methods may not be performed sequentially. Some steps are optional such that every step of the methods may not be performed.

1. A method of generating vocal data, comprising;
transmitting an acoustic waveform through air to a skin of a subject;
measuring a reflected acoustic waveform reflected by the skin of the subject; and
processing the measured reflected acoustic waveform to generate vocal data of the subject.

2. The method of claim 1, wherein the reflected acoustic waveform comprises the acoustic waveform coupled with a voice signal corresponding to a vibration in the skin.

3. The method of claim 1, wherein the reflected acoustic waveform comprises a change in one or more of a phase and a frequency relative to the acoustic waveform.

4. The method of claim 1, wherein the acoustic waveform and the reflected acoustic waveform are each configured to be immune to coupling with an external noise waveform.

5. The method of claim 4, wherein the external noise waveform comprises one or more of a sound waveform other than those produced by the subject, an external ultrasound waveform, and an external electromagnetic waveform.

6. The method of claim 1, wherein the acoustic waveform is transmitted continuously.

7. The method of claim 1, wherein the reflected acoustic waveform is measured continuously.

8. The method of claim 1, wherein the acoustic waveform is transmitted from between about 5 cm and about 15 cm away from the skin of the subject.

9. The method of claim 1, wherein the reflected acoustic waveform is measured from between about 5 cm and about 15 cm away from the skin of the subject.

10. The method of claim 1, wherein the acoustic waveform comprises a frequency of between about 40 kHz and about 300 kHz.

11. The method of claim 1, wherein the acoustic waveform comprises a field of view of between about 30 degrees and about 120 degrees.

12. The method of claim 1, wherein the acoustic waveform comprises a sound pressure level between about 90 dB and about 135 dB at about 30 cm.

13. The method of claim 1, wherein the skin of the subject comprises a skin of one or more of a neck, a jaw, and a chin.

14. The method of claim 13, wherein the skin of the neck comprises a skin covering one or more of a larynx, a circloid cartilage, a thyroid cartilage, a thyroid gland, a trachea, and a lymph node.

15. The method of claim 1, wherein processing the measured reflected waveform comprises processing a scattered acoustic waveform.

16. The method of claim 1, wherein transmitting the acoustic waveform and measuring the reflected acoustic waveform comprises using at least one transducer.

17. (canceled)

18. The method of claim 1, wherein measuring the reflected acoustic waveform comprises using at least one micro-electro-mechanical-systems (MEMS) microphone.

19. (canceled)

20. The method of claim 1, wherein processing the measured reflected acoustic waveform comprises estimating a change in one or more of a phase and a frequency of the measured reflected acoustic waveform.

21-26. (canceled)

27. The method of claim 1, further comprising inputting the vocal data to a machine learning model and generating a response using the machine learning model.

28. The method of claim 1, further comprising analyzing the vocal data using a natural language model to generate a response.

29. The method of claim 28, further comprising communicating the response of the natural language model to the subject.

30. The method of claim 1, further comprising transmitting the vocal data using a communication device to one or more of the subject, another subject, and a computing device.

31. The method of claim 1, further comprising releasably coupling a device to the subject, the device comprising a support configured to releasably couple to the subject, at least one transducer coupled to the support, and a processor and memory coupled to the support.

32. The method of claim 31, wherein the at least one transducer is between about 5 cm and about 15 cm away from the skin of the subject.

33. The method of claim 31, wherein the at least one transducer is configured to transmit the acoustic waveform at a frequency of between about 40 kHz and about 300 kHz.

34. The method of claim 31, wherein the at least one transducer is configured to transmit the acoustic waveform in a field of view of between about 30 degrees and about 120 degrees.

35. The method of claim 31, wherein the at least one transducer is configured to measure a scattered acoustic waveform.

36. The method of claim 31, wherein releasably coupling the device comprises coupling the device to one or more of a neck, a shoulder, a back, and a chest of the subject.

37. The method of claim 31, further comprising analyzing the vocal data using a natural language model to generate a response.

38. The method of claim 37, further comprising communicating the response to the subject.

39-116. (canceled)
